(19) United States
(12) Patent Application Publication
(10) Pub. No.: US 2005/0003013 A1
(43) Pub. Date: Jan. 6, 2005

(54) Drug

(76) Inventors: Nobuhiko Yui, Ishikawa (JP); Koichi Murakami, Ishikawa (JP); Tooru Ooya, Ishikawa (JP); Ikuo Sato, Kanagawa (JP); Tuyoshi Nakama, Ishikawa (JP); Ryouji Kawabata, Kanagawa (JP)

Correspondence Address: WENDEROTH, LIND & PONACK, L.L.P., 2033 K STREET N. W., SUITE 800, WASHINGTON, DC, 20006-1021, US

(21) Appl. No.: 10/829,243

(22) Filed: Apr. 22, 2004

(30) Foreign Application Priority Data
Apr. 25, 2003 (JP) 2003-122861

(51) Int. Cl. A61K 9/14

(52) U.S. Cl. 424/488

(57) ABSTRACT

A drug is provided comprising of a degradable gel with a saturated moisture content not exceeding 98 wt. % and a functional material, and which permits control of the rate of release of the functional material and performs controlled-release of the functional material over a prolonged period of time, and wherein the gel itself decomposes and dissipates upon completion of release of the functional material.

### FIELD OF INVENTION

The present invention relates to a drug and process for preparing thereof, and a method for controlling release of a functional material.

### BACKGROUND OF THE INVENTION

Drugs are extremely useful in the treatment of humans and animals or in plant and pest control to the extent that they are indispensable in modern societies. However, they also may exhibit a detrimental effect on organisms and the environment. Therefore, in order to reduce adverse effects, the goal to apply drugs precisely and efficiently has been continuous since the birth of drugs. For example, when beneficial effect over a prolonged period are desired, methods employing supported forms as drug carriers are considered.

For example, functional materials including danazol for the treatment of endometriosis and which are used as orally administered drugs at present, are known to induce adverse effects such as hepatic function disorder, increase of body weight, sterility, menstrual disorder, edema, virilization, and thrombosis. As an alternative treatment avoiding these adverse effects, intrauterine implanted drug products in which the drugs for endometriosis danazol is carried by silicone rubber or acetylhyaluronic acid (salts) have been disclosed (see patent documents 1 and 2, e.g.).

Although, where the above described intrauterine implanted drug product in which a drugs for endometriosis is carried on silicone rubber, there is apprehension that the silicone rubber, which exhibits an unstable rate of release of the functional material, may have detrimental effects on intrauterine tissues. Furthermore, since the silicone rubber remains in the uterus even after complete release of the treatment drug, removal of the carrier imposes additional physical and mental strain on patients.

When functional materials are carried by dimethyldistearylammonium salt of acetylhyaluronic acid, controlled release of functional materials is also difficult. In addition, as a gradual release method for a low water soluble functional material, the dispersing of micro-sphere functional materials in a hyaluronic acid-ethylene glycol diglycidyl ether gel is known. However, in this case, the release of functional materials was excessively rapid to achieve gradual release over a prolonged period.

Earlier literature:
[Patent document 1] Japanese patent No. 2,590,358
[Patent document 2] Japan patent laid-open 2002-356447

### PROBLEMS TO BE SOLVED BY THE INVENTION

As described above, the problems of the present invention is to provide a drug which comprises a degradable gel and a functional material, and which can control the rate of release of the functional material and perform controlled-release of the functional material over a prolonged period, and wherein the gel itself decomposes and dissipates upon completion of release of the functional material.

### MEANS FOR SOLVING THE PROBLEM

From our research, we found that a degradable gel with a saturated moisture content not exceeding 98 wt. % does not decompose readily in organisms, and that by regulating the saturated moisture content in the range not exceeding 98 wt. %, the rate of decomposition rate of the gel can be controlled. We also found that a degradable gel comprising a functional material in which a functional material is carried by a degradable gel with a saturated moisture content not exceeding 98 wt. % can control both the duration and the rate of release of the functional material, and that the gel itself decomposes and dissipates after completion of release of the functional material. The present invention is based on these findings.

The present invention is constituted as following:
{1} A drug comprising a degradable gel with a saturated moisture content not exceeding 98 wt. % and a functional material.
{2} A drug comprising a degradable gel with a saturated moisture content not exceeding 98 wt. % and a functional material, wherein the rate of release of the functional material is controlled by varying the saturated moisture content of the degradable gel.
{3} The drug as described in the item {1} or {2}, wherein the functional material is at least one selected from the group of intrauterine administered drugs, intravaginal administered drugs, intratumoral administered drugs of endometriotic cysts, and intrapelvic administered drugs.
{4} The drug as described in the item {1} or {2}, wherein the functional material is danazol.
{5} The drug as described in the item {1} or {2}, wherein the degradable gel is a polysaccharide gel.
{6} The drug as described in the item {5}, wherein the polysaccharide gel is an anionic polysaccharide gel.
{7} The drug as described in the item {1} or {2}, wherein the degradable gel is a gel obtained through a crosslinking reaction using a crosslinking agent.
{8} The drug as described in the item {7}, wherein the crosslinking agent is an epoxide displaying not less than two epoxy groups per molecule.
{9} The drug as described in the item {8}, wherein the epoxide is ethylene glycol diglycidyl ether.
{10} The drug as described in the item {1} or {2}, wherein the drug further comprises a surfactant.
{11} The drug as described in the item {10}, wherein the surfactant is a nonionic surfactant.
{12.} In a drug comprising a degradable gel and a functional material, a method for controlling release of a functional material characterized in that the rate of release is controlled by varying the saturated moisture content of the degradable gel.
{13} A process preparation for a drug comprising the following steps:
   (First step) mixing of a functional material and surfactant so as to obtain a surfactant suspension comprising the functional material;
   (Second step) dissolving the components of a degradable gel in such a proportion as to comprise 20 to 80 wt. % aqueous solvent in order to form the raw materials solution of a degradable gel; and
   (Third step) mixing the surfactant suspension comprising the functional material and the raw materials solution of a degradable gel, and adding a crosslinking agent so as to crosslink the raw materials of a degradable gel.

### DETAILED DESCRIPTION

A drug according to present invention comprises a degradable gel and a functional material, and where the degradable gel with a saturated moisture content not exceeding 98 wt. % can control both the rate and duration of release of the functional material. In addition, in a drug according to present invention, the speed of decomposition of the degradable gel is controlled by its saturated moisture content, and the quantity of release of the functional material can be controlled through the decomposition rate of the degradable gel.

In the present invention, the degradable gel is characterized by possessing a saturated moisture content not exceeding 98 wt. %, and preferably not exceeding 96 wt. %, more preferably not exceeding 93 wt. %, further preferably not exceeding 89 wt. %. The lowest limit of the saturated moisture content is not specified, however, is preferably not less than 50 wt. %, more preferably not less than 60 wt. %, further preferably not less than 70 wt. %, particularly preferably not less than 80 wt. %.

The saturated moisture content of the present invention is defined as the weight percentage of water in the gel determined from the formula, (weight of wet gel−weight of dry gel)/weight of wet gel×100, and where the weight of the wet gel is the weight of the gel at 25° C. in pure water under equilibrium conditions. The equilibrium state is the state of wet gel left in pure water for 100 hours.

The degradable gel is one which decomposes under a wet environment such as in an organism, and is either a gel consisting of a polymer compound which decomposes under the environment and crosslinking agents or a gel in which the coupling sites of the polymer compound and crosslinking agents decompose. Polymer compounds which decompose under the environment described above and can be used as raw materials of the present invention include anionic polysaccharides, cationic polysaccharides, dextrans, chitosans, ribonucleic acids, and deoxyribonucleic acids. In the present invention, anionic polysaccharides are especially preferable. The degradable gel used in the present invention may consist of a plurality of polymer compounds. And, even if crosslinking agents are used, a plurality of polymer compounds may be used.

The anionic polysaccharide is a polysaccharide possessing a negative charge as the result of a carboxyl group, sulfuric group, and salts inclusive thereof. In the present invention, the anionic polysaccharide includes cellouronic acid, salts of cellouronic acid, alginic acid, salts of alginic acid, polygalacturonic acid, salts of polygalacturonic acid, and glycosaminoglycan. Glycosaminoglycan includes heparin, salts of heparin, heparan sulfate, salts of heparan sulfate, chondroitin, salts of chondroitin, chondroitin sulfate, salts of chondroitin sulfate, dermatan sulfate, salts of dermatan sulfate, hyaluronic acid, and salts of hyaluronic acid. When these anionic polysaccharides are used in a drug as polymer compounds, the intravital decomposition of the degradable gel of the drug and subsequent release of the functional material probably correlates with the biorhythm of patients administered with the drug, and more effective medical benefits can be expected.

In the case of a drug consisting of a degradable gel being composed of the above anionic polysaccharides, especially hyaluronic acid, and salts of hyaluronic acid (hereinafter, abbreviated as “hyaluronic acid (salts)”) and intrauterine administrating drugs or intravaginal administrating drugs, high correlation between intrauterine or intravaginal decomposition of the degradable gel of the drug and following release of the functional material and biorhythm of patients administered the drug can be expected, and a degradable gel composing of hyaluronic acid (salts) can be especially preferably used in the present invention.

In the present invention, when hyaluronic acid (salts) is used as the raw material of a degradable gel, the average molecular weight of the hyaluronic acid (salts) determined by HPLC method is preferably not larger than 1000 kDa, more preferably not larger than 500 kDa, further preferably not larger than 300 kDa. If the average molecular weight is in the range, a gel with low saturated moisture content can be preferably obtained by crosslinking under the specified crosslinking condition.

When hyaluronic acid (salts) is used as the raw material of a degradable gel, at present, it is not obvious whether intrauterine or intravaginal decomposition of the degradable gel component of the drug and the following release of the functional material correlates with the biorhythm of patients administered the drug. However, in the uterus or vagina, the secretion of hyaluronic acid decomposition enzyme (hyaluronidases) and the generation of active oxygen change under the influence of the sexual cycle, and the change can be used for effective control of release. That is, since speed of decomposition of the degradable gel changes under the influence of hyaluronidases at the treatment site and active oxygen, controlled-release corresponding to surrounding concentrations of hyaluronidases and active oxygen is possible (see tables 1 and 2).

Table 1 shows the linear velocities of decomposition by hyaluronidase measured by shaking hyaluronic acid-ethylene glycol diglycidyl ether gel at 37° C. in phosphate buffer solution (0.14 mol/L, pH: 4.5) in which a predetermined amount of hyaluronidase is dissolved and weighing periodically the weight of the gel. Table 2 shows the linear velocities of decomposition by active oxygen (hydroxy radical) measured by dipping the same gel as used in table 1 in iron (II) sulfate solution (50 mmol/L) for two days, then dipping in a predetermined concentration of hydrogen peroxide solution in which active oxygen (hydroxy radical) is generated on the gel surface and weighing the weight of the gel.

Release duration of a functional material (medical benefits duration) may be determined on an as needed basis. However, release duration can be fundamentally regulated by decomposition rate of a degradable gel used in the present invention, surface area and volume of the drug. Decomposition rate of a degradable gel can be controlled by regulating parameters such as type, chemical structure, three-dimensional structure and molecular weight of a degradable gel. For example, when using hyaluronic acid (salts) gel, by selecting its saturated moisture content, the decomposition rate can be controlled and release duration can be arbitrarily established (see FIG. 2).

When the functional material carried by anionic polysaccharide displays low solubility in water such as danazol described below, its elution from the carrier can be prevented by preparing a suspension with a surfactant and combining it into the carrier. Association with the carrier, if the functional material is ionic, proceeds through ionic coupling with the degradable gel, and moreover, where the functional material possesses functional groups, proceeds with covalent bonding.

The origin of hyaluronic acid (salts) used in the present invention is not specified, and may be derived from tissues of animals such as comb or microorganisms with the capacity to produce hyaluronic acid (salts). However, it is preferable that hyaluronic acid (salts) used in the present invention is derived from microorganisms.

A drug of the present invention, in the preparation process, includes a surfactant, and the preparation process is classified into three main steps as follows:
(First step) mixing a functional material and surfactant to obtain a surfactant suspension comprising the functional material;
(Second step) dissolving the components of a degradable gel in such a proportion as to comprise a 20 to 80 wt. % aqueous solvent in order to form the raw materials solution of a degradable gel; and
(Third step) mixing the surfactant suspension comprising the functional material and the raw materials solution of a degradable gel, and adding a crosslinking agent so as to crosslink the raw materials of a degradable gel.

The first step is explained in detail using danazol as an example, as follows.
After the surfactant is dispersed in pure water, a gynopathy treatment drug, danazol is added and the suspension is created with a homogenizer. Selection of a nonionic surfactant is preferable, in particular, such as Polysolvate 80, Polyoxyethylene (20) sorbitan monolaurate and sucrose stearate. The mixing ratio of the gynopathy treatment drug to surfactant is preferably in the proportion of 1 weight part gynopathy treatment drug to 1/100 to 10 weight parts surfactant, more preferably 1/20 to 1 weight parts surfactant. The concentration of danazol in the surfactant suspension comprising the gynopathy treatment drug obtained by mixing both in these ratios can be arbitrarily selected by back calculation from the amount of danazol present in the final state of the gynopathy treatment drug. The concentration of danazol is not specified, however, is preferably from 0.01 to 30 wt. %, more preferably from 10 to 20 wt. %. In the present invention, pure water equates to refined water by, for example, continuous ion exchange (Electric Deionization), reverse osmosis etc.

The second step is explained in detail using hyaluronic acid (salts) as the raw material of a degradable gel, as follows.
Hyaluronic acid (salts) gel solution is prepared by the addition of hyaluronic acid (salts) in the proportion of 20 to 80 wt. % to sodium hydroxide aqueous solution and mixing homogeneously.

The third step is explained in detail, as follows.
The suspension and the hyaluronic acid (salts) gel solution are mixed by stirring with a spatula until homogeneous. An epoxy compound is then added and stirred again with a spatula. The obtained viscous solution is rapidly cast into a mold and heated in a thermostatic chamber. By neutralizing the obtained gel with hydrochloric acid aqueous solution (0.05 mol/L) and washing with pure water for 24 hours, a hyaluronic acid (salts)-epoxy compound gel comprising danazol is obtained.

The concentration of the sodium hydroxide aqueous solution must be sufficient to induce full crosslinking of the epoxy compound having not less than two epoxy groups per molecule and to dissolve the hyaluronic acid (salts), is preferably from 0.01 to 10 mol/L, more preferably from 0.1 to 5 mol/L. The mixing ratio of the danazol suspension to the sodium hydroxide aqueous solution can be arbitrarily selected by back calculation from the amount of danazol in the final state of the gynopathy treatment drug. The mixing ratio of the danazol suspension to the sodium hydroxide aqueous solution is preferably in the proportion of 1 to 10000 to 9 to 1 (volumetric ratio). The concentration of hyaluronic acid (salts) in the sodium hydroxide aqueous solution comprising hyaluronic acid (salts) is not specified, however, is preferably not less than 10 wt. %, more preferably not less than 20 wt. %.

If the degradable gel in the present invention is a gel obtained by crosslinking with a crosslinking agent, the crosslinking agent should possess not less than two epoxy groups per molecule. Crosslinkable compounds with hydroxy groups of hyaluronic acid are epoxy compounds having not less than two epoxy groups per molecule such as ethylene glycol diglycidyl ether, polyethylene glycol diglycidyl ether, epichlorohydrin, trimethylolpropane polyglycidyl ether, neopentyl glycol diglycidyl ether, glycerol polyglycidyl ether, polypropylene glycol diglycidyl ether and sorbitol polyglycidyl ether, and preferably ethylene glycol diglycidyl ether. Incidentally, the addition of a crosslinking agent is preferably from 0.01 to 10 equivalents, more preferably from 0.05 to 5 equivalents to the reactive functional group with the crosslinking agent.

A drug in the present invention may comprise, other than the functional materials, in the range without loss of effects of the present invention sugars, amino acids, peptides, proteins, enzymes, lipids, inorganic salts, organic salts, metals, etc.

Danazol used in the present invention may be analyzed by the HPLC method. For example, Zorbax CN (trade name, manufactured by Shimadzu GLC) or Lichrosorb RP-18 (trade name, manufactured by Merck) may be used. In the former case, it is preferable to analyze under the following conditions (eluent: mixing solvent of methanol, acetonitrile, and water (mixing volume ratio; 3:2:7), elution rate: 1 mL/min, column temperature: 30° C.). In the latter case, it is preferable to analyze under the following conditions (eluent: mixing solvent of methanol, and water (mixing volume ratio; 8:2), elution rate: 1 mL/min, column temperature: 25° C.). In both cases, it is preferable to detect and quantify by absorbance measurement at UV 260 nm.

When a drug of the present invention is intended for implantation in the uterus, vagina, tumor of endometriotic cyst, or pelvis, the shape and size of a drug of the present invention is not specified, so long as it is suitable for intrauterine, intravaginal, intratumor of endometriotic cyst, or intrapelvic topical administration. In relation to the shape, in the case of using an intrauterine treatment drug of the present invention in the uterus, a form such as a T-shape, Ohta ring-like, sheet-like gel, spherical gel may be applied, and in the case of using in the vagina, a form such as a circular ring may be applied. In addition, in the case of application in tumor of endometriotic cyst, or pelvis, a form such as a fluid gel or sheet-like gel may be applied.

The dimensions of the present invention may change depending on the application objectives. When an administration drug is a T-shaped intrauterine treatment drug, the length along the cross axis is preferably from 20 to 40 mm, more preferably from 30 to 35 mm, the length along the vertical axis is preferably from 25 to 45 mm, more preferably from 30 to 38 mm, and its diameter is preferably from 3.0 to 4.0 mm, more preferably from 3.2 to 3.6 mm.

In the case of an Ohta ring-like intrauterine treatment drug, the ring outer diameter is preferably from 20 to 25 mm, and the ring thickness is preferably from 2.55 to 4.5 mm, more preferably about 3.0 mm. In the case of a sheet-like gel, the cross directional length is preferably from 10 to 50 mm, more preferably from 20 to 30 mm, the length in the longitudinal direction is preferably from 20 to 70 mm, more preferably from 40 to 60 mm, and the thickness is preferably from 2 to 20 mm, more preferably from 5 to 10 mm.

In the case of a spherical gel, the diameter is preferably from 10 to 30 mm, more preferably from 20 to 25 mm. In the case of a circular ring intravaginal treatment drug, the ring outer diameter is preferably from 30 to 60 mm, more preferably from 45 to 55 mm, and the ring diameter is preferably from 4.0 to 12.0 mm, more preferably from 7.5 to 10.0 mm. Also, in the case of a paste-like gel, the size is not specified.

When a drug of the present invention is in the form of a T-shape, stick-like, or Ohta ring-like, the structure may not be only limited to a single layer, but also, so as to increase the hardness of the drug, may be multi layered of not less than two layers with an embedded core consisting of plastic etc.

However, for a T-shaped intrauterine treatment drug containing an embedded core, embedding is usually carried out in both the cross axis and vertical axis, and it is preferable that the lengths of the core are range from 55 to 70% of the axis dimensions, and the diameter the of core is in the range from 60 to 90% of the axis diameter. Moreover, the drug is preferably fixed along the vertical axis. Furthermore, in a T-shaped intrauterine treatment drug, it is preferable that a nylon mono filament, with a length of preferably from 30 to 400 mm, more preferably from 50 to 280 mm, and of a diameter preferably from 0.170 to 0.290 mm, is attached to the bottom end of the vertical axis.

In a stick-like intrauterine treatment drug, when core is embedded, it is preferable that the length of core is in the range of from 55 to 70% of the stick length, and the diameter of core is in the range of from 60 to 90% of the stick diameter. Furthermore, in a stick-like intrauterine treatment drug, it is preferable that nylon mono filament, of which length is preferably from 30 to 400 mm, more preferably from 50 to 280 mm, and of which diameter is preferably from 0.170 to 0.290 mm, is attached to the bottom end.

Also, when forming a drug of the present invention as a circular ring, so as to increase the rate of release of the functional material in response to treatment duration or severity of symptoms, the drug may be formed as either single or multi layers. However, when forming a double-layered circular ring intravaginal drug, the thickness of the upper layer is preferably at least 0.1 mm, more preferably from 0.1 to 2.0 mm.

A double-layered drug is prepared in the same process as that of a single layer drug described above, with the additional embedding of the desired core in the casting step followed by solidification in the same way. The core described above is used. When a drug of the present invention is used in organisms, the drug is required to be aseptic. Therefore, it is important that the drug is maintained under aseptic conditions during preparation and final packaging using as such aluminum heat-sealed packages.

Functional materials used in the present invention, not being specified, are medicinal properties relating to intrauterine administration drugs, intravaginal administration drugs, intratumoral administration drugs of endometriotic cyst, and intrapelvic administration drugs, etc. Cited examples of medicinal properties are, for example, drugs for treatment of endometriosis, contraceptives, antipyretics, hormonal drugs, drugs for treatment of endometrial cancer, inhibitor of hormone synthesis use for treatment of endometriosis, antibiotics, antifingals, drugs for treatment of colpitis, drugs for treatment of trichomoniasis, drugs for treatment of uterine cervical cancer. When the medicinal properties are related to drugs for treatment of endometriosis, effects of the present invention are remarkable.

A drug for treatment of endometriosis is, for example, danazol, nonsteroidal anti-inflammatory drugs, herbal medicines, progestogen, estrogen, GnRH-antagonists, gienogest, angiogenesis inhibitors, aromatase inhibitors. Most significant is, danazol, which can be expected to present remarkable medical benefits in topical administration, can be preferably used in the present invention.

When the medicinal properties of a drug of the present invention is danazol, the preferable raw material for a degradable gel is hyaluronic acid (salts) and its derivatives. Wherever these compounds are used, danazol is favorably carried by the degradable gel, and the degradable gel responds favorably to hyaluronidases and active oxygen, allowing danazol to be released instantaneously.

When the medicinal properties of a drug of the present invention is an drugs for treatment of endometriosis, the drug can be used in treating either intrinsic or extrinsic endometriosis.

When the medicinal properties are drugs of intrauterine administration, drugs of intravaginal administration, drugs of intratumoral administration of endometriotic cyst, or drugs of intrapelvic administration, the applicability of the drug is not limited to human females, but may also be applied to mammalia such as pig, cow, horse, sheep, dog, cat, and monkey.

The applicable field of a drug of the present invention is not specified, and can be used in fields such as medicine, food, agriculture, and hygienics. The drug can be used in, for example, DDS (Drug Delivery System) in the medical field, NDS (Nutrient Delivery System) or controlled-release of a preservative and quality maintenance improver in the food field, as agricultural chemicals and fertilizers in agriculture, and controlled-release of antiseptics, antibacterial agent, fungicides or funginerts used in pools, water tanks, bathtubs in hygiene.

### EXAMPLE

The present invention is explained in particular with examples, as follows.

Measurement of Average Molecular Weight by HPLC Method

Any columns suitable for molecular weight measurement of polysaccharides can be utilized. When the polysaccharide is hyaluronic acid (salts), it is preferable to utilize columns, for example, such as Shodex Ionpak KS806 (trade name, manufactured by SHOWA DENKO K. K.), Ionpak KS-G (trade name, manufactured by SHOWA DENKO K. K.). In examples and comparative examples of the present invention, Shodex Ionpak KS806 (trade name, manufactured by SHOWA DENKO K. K.) and Ionpak KS-G (trade name, manufactured by SHOWA DENKO K. K.) were utilized. In these cases, as eluent, sodium chloride aqueous solution (0.2 mol/L) was used. The elution rate was 1 mL/min. Hyaluronic acid (salts) was detected at 206 nm. Average molecular weight can be obtained by calculation utilizing a calibration curve based on ultimate viscosities of sodium hyaluronate with known molecular weights.

Preparation of Danazol Suspension

By dispersing specified amounts of Polysolvate 80 in pure water (5 mL), then adding specified amounts of danazol and stirring with a homogenizer (Labo-Disperser X10/25, Genarater shaft 10F, trade name, manufactured by IUCHI SEIEIDO Co.) at the rate of 24000 rpm for one minute, danazol suspension A, B, and C were obtained. Individual amounts of danazol and Polysolvate 80 in suspension A, B, and C was as follows.

Danazol suspension A (12 wt. % of danazol): danazol (600 mg), Polysolvate 80 (150 mg); Danazol suspension B (24 wt. % of danazol): danazol (1200 mg), Polysolvate 80 (300 mg); Danazol suspension C (1.2 wt. % of danazol): danazol (60 mg), Polysolvate 80 (15 mg)

### Examples 1 to 4

Preparation of Danazol-Carried Hyaluronic Acid-Ethylene Glycol Diglycidyl Ether Gel (Hereinafter, Abbreviated as “D-CHA-EGDGE Gel”) for In Vitro Testing

Either one (2.5 mL) of danazol suspension A or danazol suspension B was mixed with sodium hydroxide aqueous solution (2 mol/L, 2.5 mL), then sodium hyaluronate (average molecular weight of 90 kDa, CHA manufactured by CHISSO Co. (hereinafter, abbreviated as “CHA”), 1500 mg) was added and stirred. In addition, ethylene glycol diglycidyl ether (870 mg) was added and stirred, and cast into a mold with predetermined form, then heated in a thermostatic chamber (80° C.), for 15 min in EXAMPLE 1, for 14 min in EXAMPLE 2, for 20 min in EXAMPLES 3 and 4.

Gel extracted from the mold was neutralized with hydrochloric acid aqueous solution (0.05 mol/L) and washed with pure water, dipped in phosphate buffer solution (pH 4.5, 0.14 mol/L) for two days, then cut into the predetermined form (disc-like, diameter; 10 mm, thickness; about 2 mm) to obtain the danazol-carried hyaluronic acid gel. The danazol content and saturated moisture content of the obtained danazol-carried hyaluronic acid gels are shown, as follows.
EXAMPLE 1: danazol suspension A, danazol content (1.4 mg), saturated moisture content (92 wt. %)
EXAMPLE 2: danazol suspension A, danazol content (0.8 mg), saturated moisture content (94 wt. %) EXAMPLE 3: danazol suspension B, danazol content (3.0 mg), saturated
moisture content (89 wt. %)
EXAMPLE 4: danazol suspension A, danazol content (1.6 mg), saturated moisture content (89 wt. %)

### Comparative Example 1

Preparation of D-CHA-EGDGE Gel for In Vitro Testing

Danazol suspension B (2.5 mL) was mixed with sodium hydroxide aqueous solution (2 mol/L, 2.5 mL), then sodium hyaluronate (average molecular weight of 1000 kDa, CHA manufactured by CHISSO Co. (hereinafter, abbreviated as “CHA”), 750 mg) was added and stirred. In addition, a mixed solution of ethylene glycol diglycidyl ether (435 mg) and ethanol (0.1 mL) was added and stirred, and cast into a mold of predetermined form, then heated in a thermostatic chamber (60° C.) for 15 min.

Gel extracted from the mold was neutralized with hydrochloric acid aqueous solution (0.05 mol/L) and washed with pure water, dipped in phosphate buffer solution (pH 4.5, 0.14 mol/L) for two days, then cut into a predetermined form (disc-like, diameter; 10 mm, thickness; about 2 mm) to obtain the danazol-carried hyaluronic acid gel. The danazol content and saturated moisture content of the obtained danazol-carried hyaluronic acid gel is shown, as follows.

COMPARATIVE EXAMPLE 1: danazol suspension B, danazol content (0.16 mg), saturated moisture content (99.5 wt. %)

### Examples 5 and 6

Preparation of D-CHA-EGDGE Gel for In Vivo Testing (Rat Intrauterine Indwelling Gel)

Either one (2.5 mL) of danazol suspension A or danazol suspension C was mixed with sodium hydroxide aqueous solution (2 mol/L, 2.5 mL), then CHA (average molecular weight of 90 kDa, 1500 mg) was added and stirred. In addition, ethylene glycol diglycidyl ether (870 mg) was added and stirred, and cast into a mold of predetermined form, then heated in a thermostatic chamber (80° C.) for 15 min.

Gel extracted from the mold was neutralized with hydrochloric acid aqueous solution (0.05 mol/L) and washed with pure water, dipped in phosphate buffer solution (pH 4.5, 0.14 mol/L) for two days to obtain the danazol-carried hyaluronic acid gel with 90 wt. % of saturated moisture content. The danazol content and saturated moisture content of the obtained danazol-carried hyaluronic acid gels are shown, as follows. EXAMPLE 5: danazol suspension A, danazol content (1 mg), saturated moisture content (90 wt. %), shape; stick-like gel (length; 20 mm, diameter; 2 mm, inside diameter; 0.7 mm) supporting inserts consisting of plastics (diameter; 0.7 mm, length; 22 mm) EXAMPLE 6: danazol suspension C, danazol content (0.1 mg), saturated moisture content (90 wt. %), shape; stick-like gel (length; 20 mm, diameter; 2 mm, inside diameter; 0.7 mm) supporting inserts consisting of plastics (diameter; 0.7 mm, length; 22 mm)

### Comparative Examples 2 and 3

Preparation of Danazol-Carried Dimethyldistearylammonium Salt of Acetylhyaluronic Acid (Hereinafter, Abbreviated as “D-AcCHA-DSC”)

Sodium hyaluronate (average molecular weight of 1000 kDa, CHA manufactured by CHISSO Co. (hereinafter, abbreviated as “CHA”), 5.4 g) was dissolved in pure water (900 mL), and dimethyldistearylammonium chloride (hereinafter, abbreviated as “DSC”, 7.41 g) was suspended in pure water (1680 mL). Both liquid were heated up to 45° C., mixed with stirring and stirred for 5 min. Prepared complex was separated by centrifuging (5000 rpm, at room temperature) and washed with warm water (at 40° C.). After washing, the complex was freeze-dried over night, and another vacuum-dried over night at 50° C., and CHA-DSC complex was obtained (yield; 9.9 g, yield percentage; 85%). The CHA-DSC complex (9.0 g) was dissolved in a mixed solvent of DMF (300 mL) and acetyl chloride (2.4 g in Comparative Example 2, 1.2 g in Comparative Example 3) and pyridine (2.4 g in Comparative Example 2, 1.2 g in Comparative Example 3) and stirred for two hours at 60° C. By adding water (1.5 L) under cooling in an ice-bath, filtering the gel-like material, washing with water, and vacuum-drying over night at 50° C., AcCHA-DSC (8.5 g in Comparative Example 2, 7.0 g in Comparative Example 3) was obtained.

Then, danazol (0.05 g in Comparative Example 2, 0.025 g in Comparative Example 3) and AcCHA-DSC (0.75 g) were added to pure water (2 mL) and the suspensions were immediately cast in a predetermined mold (disc-like, diameter; 10 mm, thickness; 2 mm), and freeze-dried to obtain D-AcCHA-DSCs. Individual weights of obtained D-AcCHA-DSCs were 100 mg.

The danazol content of the D-AcCHA-DSCs are shown, as follows.
COMPARATIVE EXAMPLE 2: danazol content (5 mg)
COMPARATIVE EXAMPLE 3: danazol content (2.5 mg)

Test of Decomposition of D-CHA-EGDGE Gel and D-AcCHA-DSC by Hyaluronidase and Release of Danazol

Each of D-CHA-EGDGEs gel (Examples 1 to 4, Comparative Example 1) and D-AcCHA-DSCs (Comparative Examples 2 and 3) was shaken in 25 mL of phosphate buffer solution (comprising hyaluronidase (derived from cow testicle, Type IV-S, manufactured by SIGMA Co., 10 unit/mL), pH 4.5, 0.14 mol/L). Phosphate buffer solution comprising hyaluronidase was renewed periodically, the change of gel weight was measured and danazol concentration in Phosphate buffer solution was measured by HPLC. FIG. 2, in which the cumulative decomposition amounts of the gel are plotted with time, shows that these gels decompose linearly in relation with time. FIG. 2 also shows that the gel of Comparative Example 1 decomposed and dissipated in about two days, while, individual decomposition times of these gels of Example 1 to 4 having the same shape and dimensions differs from 10 to 80 days. In addition, in Comparative Examples 2 and 3, weight loss of about 15% was found in 30 days, therefore, there was no difference depending on the addition amount of acetyl chloride. FIG. 3, in which the cumulative releasing amounts of danazol are plotted with time, shows that Examples 1 to 4 are controlled in a larger range of danazol release and release duration than that of Comparative Examples 1 and 2, since decomposition rate of Examples 1 to 4 can be controlled by their saturated moisture content. FIG. 4, in which the relation between decomposition of gel and danazol release is illustrated, shows that a corresponding amount of danazol is released to the amount of gel decomposition.

Test of D-CHA-EGDGE Gel Indwelled to Rat Uterine Cavity

D-CHA-EGDGE gel for rat intrauterine indwelling test (Examples 5 and 6) and blank danazol carried CHA-EGDGE gel as control were implanted in the uterus of rats affected by uterine adenomyosis. Extraction was performed to weigh the gel and observe the affected part after one week, two weeks, three weeks, and four weeks, respectively. Table 3 shows the results. It is shown that decomposition of the gel of Example 5 with a high danazol content is slow, considered due to the generation of hyaluronidase, and endometrium tissues were atrophied by pharmacological function of danazol. That is, it is shown that a drug of the present invention serves as an intelligent drug allowing the sufficient release of danazol when symptoms of uterine adenomyosis are severe, and on the other hand, reduce the release of danazol by inhibition of CHA-EGDGE gel decomposition when abated. Changes of endometrium tissues seen in pregnancy in Example 6, and changes of tissues caused by pharmacological function of danazol in Example 5 were found.

TABLE 1

| Saturated moisture | Concentration of | Linear velocity of |
|---|---|---|
| content | hyaluronidase | decomposition |
| (%) | (unit/mL) | (mm/day) |
| 93.5 | 100 | 0.56 |
| | 10 | 0.052 |

TABLE 2

| Saturated | Concentration | | |
|---|---|---|---|
| moisture | of iron | Concentration of | Linear velocity of |
| content | (II) sulfate | hydrogen peroxide | decomposition |
| (%) | (mmol/L) | (mmol/L) | (μm/min) |
| 86.6 | 50 | 1 | 3.5 |
| | 50 | 5 | 4.7 |

TABLE 3

| | Amount of danazol carried (mg) | | | | |
|---|---|---|---|---|---|
| | After | After | After | After | |
| | one | two | three | four | Observation of the |
| | week | weeks | weeks | weeks | affected part |
| Blank danazol | 15 | 0 | 2 | 5 | None of difference |
| carried gel | | | | | |
| (control) | | | | | |
| Example 5 | 51 | 32 | 13 | 11 | Changes of tissues |
| | | | | | caused by |
| | | | | | pharmacological |
| | | | | | function of danazol |
| | | | | | were found. |
| Example 6 | 30 | 20 | 0 | 7 | Decidual changes of |
| | | | | | endometrium |
| | | | | | (observed at |
| | | | | | pregnancy) were |
| | | | | | found. |

Initial weight was about 50 mg.

### EFFICACY OF THE INVENTION

As shown above, a drug according to the present invention can provide a drug which can control the releasing rate of the functional material and perform controlled-release of the functional material in a long term, and the gel itself decomposes and vanishes after completion of release of the functional material. When using hyaluronic acid gel, by selecting its saturated moisture content, the decomposition rate of the gel at the affected part with hyaluronidase can be controlled in a wide range, and effective releasing of the functional material can be performed.

### BRIEF DESCRIPTION OF THE DRAWINGS

FIG. 1 is a graph showing the relationship between the saturated moisture content of CHA-EGDGE gel and the linear velocity of decomposition.

FIG. 2 is a graph showing the variation per hour (cumulative value) of the decomposition amount of D-CHA-EGDGE gel.

FIG. 3 is a graph showing the variation per hour (cumulative value) of the danazol release amount of D-CHA-EGDGE gel.

FIG. 4 is a graph showing the relationship between the decomposition amount of D-CHA-EGDGE gel and the danazol release amount.

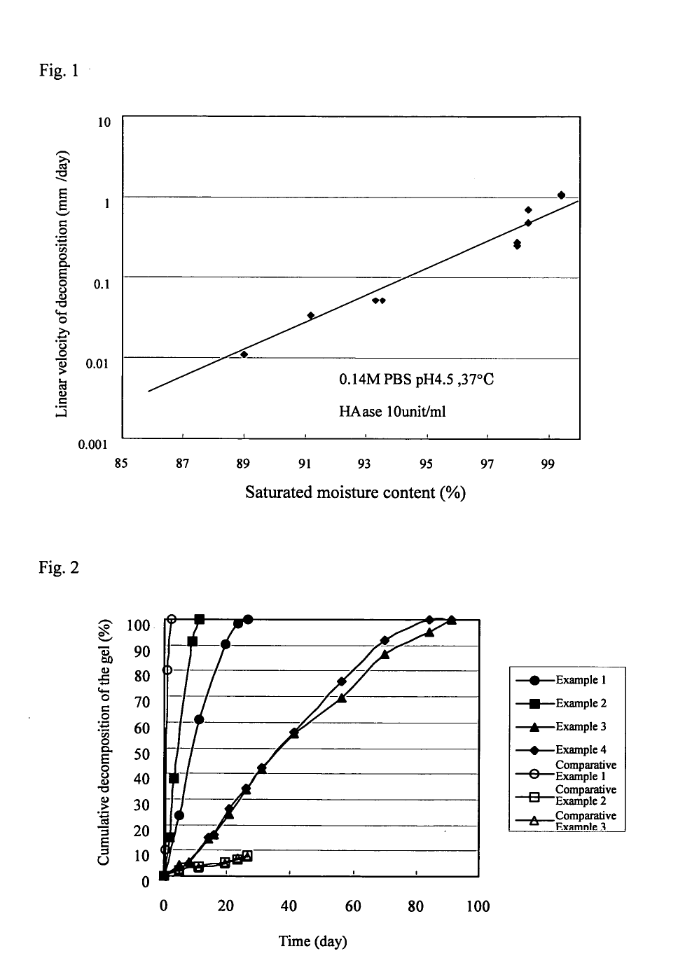

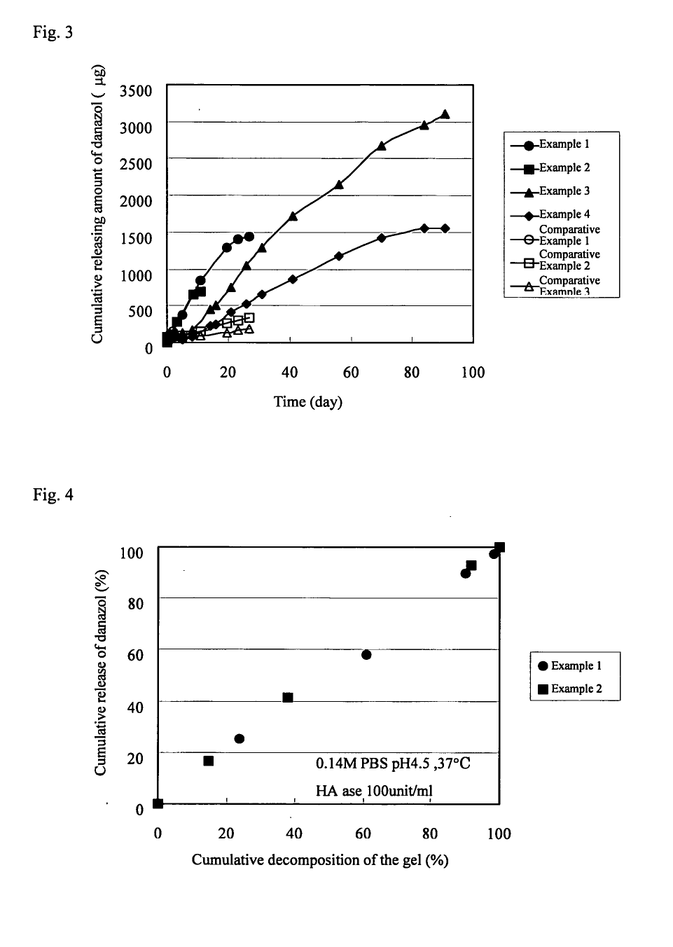

1. A drug comprising a degradable gel with a saturated moisture content not exceeding 98 wt. % and a functional material.

2. A drug comprising a degradable gel with a saturated moisture content not exceeding 98 wt. % and a functional material, wherein the release rate of the functional material is controlled by controlling the saturated moisture content of the degradable gel.

3. The drug according to claim 1, wherein the functional material is at least one selected from the group of intrauterine administration drugs, intravaginal administration drugs, intratumoral administration drugs of endometriotic cyst, and intrapelvic administration drugs.

4. The drug according to claim 2, wherein the functional material is at least one selected from the group of intrauterine administration drugs, intravaginal administration drugs, intratumoral administration drugs of endometriotic cyst, and intrapelvic administration drugs.

5. The drug according to claim 1, wherein the functional material is danazol.

6. The drug according to claim 2, wherein the functional material is danazol.

7. The drug according to claim 1, wherein the degradable gel is a polysaccharide gel.

8. The drug according to claim 2, wherein the degradable gel is a polysaccharide gel.

9. The drug according to claim 7, wherein the polysaccharide gel is an anionic polysaccharide gel.

10. The drug according to claim 8, wherein the polysaccharide gel is an anionic polysaccharide gel.

11. The drug according to claim 1, wherein the degradable gel is a gel obtained through crosslinking reaction using a crosslinking agent.

12. The drug according to claim 2, wherein the degradable gel is a gel obtained through crosslinking reaction using a crosslinking agent.

13. The drug according to claim 11, wherein the crosslinking agent is an epoxy compound having not less than two epoxy groups per molecule.

14. The drug according to claim 12, wherein the crosslinking agent is an epoxy compound having not less than two epoxy groups per molecule.

15. The drug according to claim 13, wherein the epoxy compound is ethylene glycol diglycidyl ether.

16. The drug according to claim 14, wherein the epoxy compound is ethylene glycol diglycidyl ether.

17. The drug according to claim 1, wherein the drug further comprises a surfactant.

18. The drug according to claim 2, wherein the drug further comprises a surfactant.

19. The drug according to claim 17, wherein the surfactant is a nonionic surfactant.

20. The drug according to claim 18, wherein the surfactant is a nonionic surfactant.

21. In a drug comprising a degradable gel and a functional material, a method for controlled release of a functional material characterized in that the rate of release is controlled by varying the saturated moisture content of the degradable gel.

22. A preparation process for a drug comprising the steps of:
(First step) mixing a functional material and surfactant so as to obtain a surfactant suspension comprising the functional material;
(Second step) dissolving the components of a degradable gel in such proportion to yield a 20 to 80 wt. % aqueous solvent so as to prepare the raw materials solution of a degradable gel; and
(Third step) mixing the surfactant suspension comprising the functional material and the raw materials solution of a degradable gel, and adding a crosslinking agent so as to crosslink the raw materials of a degradable gel.
